(19) United States
(12) Patent Application Publication
(10) Pub. No.: US 2006/0136016 A1
(43) Pub. Date: Jun. 22, 2006

(54) Synchronization method and apparatus and location awareness method and apparatus in chaotic communication system

(75) Inventors: Nam-hyong Kim, Seoul (KR); In-hwan Kim, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 11/304,882

(22) Filed: Dec. 16, 2005

(30) Foreign Application Priority Data
Dec. 16, 2004 (KR) 10-2004-0107164

(51) Int. Cl. A61N 1/08

(52) U.S. Cl. 607/60

(57) ABSTRACT

A synchronization method and apparatus and a location awareness method and apparatus in a chaotic communication system are provided. The location awareness method includes receiving a data frame which includes a template chaotic signal to be determined to be arranged at a predetermined position of the data frame, and a data source signal obtained by modulating the template chaotic signal, the position allocated to a user; detecting a position of the data frame in which the template chaotic signal is substantially arranged; and determining the distance between a device transmitting the data frame and a device receiving the data frame using the difference between the position of the data frame in which the template chaotic signal is substantially arranged and the predetermined position. Accordingly, it is possible to precisely detect the distance between devices.

### CROSS-REFERENCE TO RELATED PATENT APPLICATIONS

This application claims priority from Korean Patent Application No. 10-2004-0107164, filed on Dec. 16, 2004, in the Korean Intellectual Property Office, the disclosure of which is incorporated herein in its entirety by reference.

### BACKGROUND OF THE INVENTION

1. Field of the Invention

Apparatuses and methods consistent with the present invention relate to a chaotic communication system, and more particularly, to synchronization and location awareness in a chaotic communication system.

2. Description of the Related Art

Conventional wireless standards require more power consumption and complicated system construction to transmit data at high speed. However, most of industrial or home wireless monitoring and control applications require a communication system that has a longer battery life and a less complicated structure even if data transmission speed is slow.

IEEE 802.15.3a is a standard for ultra-wide band (UWB)-based wireless multimedia communications, led by the IEEE 802.15 working group. The IEEE 802.15.3a standard supports data rates up to 54 Mbps, which are still faster than those provided by competing standards such as IEEE 8012.11× or Bluetooth. Accordingly, the IEEE 802.15.3a standard is available for transmitting a digital image or multimedia data. Further, although the distance of data transmission supported by the IEEE 802.15.3a is short, i.e., up to 10 m, it is possible to connect a personal computer to various types of peripheral devices, e.g., a personal digital assistant (PDA), a mobile phone, a digital television, a set top box, a digital camera, or a game device. That is, use of the IEEE 802.15.3a standard which is a UWB-based standard, limits the range of a radio wave, thus requiring a small amount of power. Accordingly, the IEEE 802.15.3a can be applied to portable devices, and makes it possible to transmit data at low costs.

In this connection, much attention has been paid to a chaotic communication system, which is the focus the IEEE 802.15.4a standard (ZigBee Alternative PHY) extended from the IEEE 802.15.3a standard. The chaotic communication system has a simple construction and requires a small amount of power although it cannot transmit data at high speed.

Information output from the chaotic communication system is transmitted from a transmitting side to a receiving side, using a chaotic signal. Advantages of the chaotic communication system are apparent from the characteristics of the chaotic signal. The chaotic signal, which has a broadband continuous spectrum, is very sensitive to initial conditions, and can be generated by a circuit which has a simple construction and stable transmission characteristics and is manufactured at low costs. Thus, the chaotic communication system having simple RF processing is proper as a UWB-based low data rate system which requires a tradeoff between complexity and throughput.

In the chaotic communication system operating at the UWB, the range of communications is very limited, and thus, it is important to precisely recognize the location of a target device. For precise location recognition, it is required to synchronize a clock signal output from a device over a system with a clock signal output from the target device, and further, precisely detect the distance between the these devices.

### SUMMARY OF THE INVENTION

The present invention provides a synchronization method and apparatus for precise location awareness and a location awareness method and apparatus in a chaotic communication system.

According to an aspect of the present invention, there is provided a synchronization method which is performed by a synchronization apparatus which synchronizes a device over a chaotic communication system, the synchronization method comprising calculating the distance between the synchronization apparatus and the device using time when a packet is transmitted to the device and time when a packet containing a device time counter value in response to the packet is received from the device; computing an offset by comparing time when the packet containing the time counter value is to be received from the device according to the distance with the time counter value; and providing one of the offset and a (−) offset to the device so that the device adjusts the time counter value using the provided offset.

During computing the offset, the offset may be obtained by subtracting the device time counter value from the sum of a time counter value determined when the packet is transmitted and the distance.

According to another aspect of the present invention, there is provided a synchronization method in a device in a chaotic communication system, comprising receiving a packet from a coordinator; transmitting a device time counter value, which is determined when the device receives the packet, to the coordinator; receiving a computed offset from the coordinator, the computed offset being obtained by comparing time when the coordinator is to receive the device time counter value according to a distance between the device and the coordinator with the device time counter value, using time when the coordinator transmits the packet to the device and time when the coordinator receives the device time counter value; and adjusting the device time counter value by the offset.

According to another aspect of the present invention, there is provided a location awareness method in a chaotic communication system, comprising receiving a data frame which includes a template chaotic signal to be determined to be arranged at a predetermined position of the data frame, and a data source signal obtained by modulating the template chaotic signal, the position allocated to a user; detecting a position of the data frame in which the template chaotic signal is substantially arranged; and determining the distance between a device transmitting the data frame and a device receiving the data frame using the difference between the position of the data frame in which the template chaotic signal is substantially arranged and the predetermined position.

Detecting the position of the template chaotic signal comprises detecting the template chaotic signal from the data frame; performing a multiplication operation on the detected template chaotic signal and at least one data source signal, and adding the results of multiplication; and detecting from the result of addition the position of the data frame in which the template chaotic signal is substantially arranged.

According to yet another aspect of the present invention, there is provided a synchronization apparatus in a chaotic communication system, comprising a distance calculator computing the distance between the synchronization apparatus and a device, using time when a predetermined packet is transmitted to the device and time when a packet containing a device time counter value in response to the packet is received from the device; an offset calculator calculating an offset by comparing time when the device is to receive the packet containing the device time counter value according to the distance with the time counter value; and an offset transmitting unit providing one of the offset or a (−) offset to the device so that the device adjusts the time counter value by the provided offset.

The offset calculator computes the offset by subtracting the device time counter value from the sum of a time counter value determined when the packet is transmitted to the device and the distance.

According to still another aspect of the present invention, there is provided a synchronization apparatus in a chaotic communication system, comprising a packet transmitting unit transmitting a packet containing a device time counter value determined when a device receives a predetermined packet from a coordinator in response to the packet; and an offset adjusting unit receiving an offset from the coordinator and adjusting the device time counter value by the offset, wherein a distance is computed using time when the coordinator transmits the packet to the device and time when the coordinator receives the device time counter value from the device, and the offset is obtained by comparing time when the coordinator is to receive the packet containing the device time counter value according to the distance and the device time counter value.

According to still another aspect of the present invention, there is provided a location awareness apparatus in a chaotic communication system, comprising a data frame receiving unit receiving a data frame which contains a template chaotic signal to be defined to be arranged at a predetermined position of the data frame, and a data source signal obtained by modulating the template chaotic signal, the position being allocated to a user; and a signal processor detecting a position of the data frame in which the template chaotic signal is substantially arranged and determining the distance between a device receiving the data frame and a device transmitting the data frame by computing the difference between a position of the data frame in which the template chaotic signal is substantially arranged and the predetermined position.

The signal processor may include a delay circuit detecting the template chaotic signal from the data frame; a multiplier performing a multiplication operation on the detected template chaotic signal and at least one data source signal; an adder adding all the results of multiplication output from the multiplier; and a signal detector detecting, from the result of addition, the position of the data frame in which the template chaotic signal is substantially arranged.

### BRIEF DESCRIPTION OF THE DRAWINGS

The above and other aspects of the present invention will become more apparent by describing in detail exemplary embodiments thereof with reference to the attached drawings in which:

FIGS. 1A to 1C illustrate the structure of a data frame used in a chaotic communication system according to an exemplary embodiment of the present invention;

FIG. 2 is a reference diagram of a synchronization method according to an exemplary embodiment of the present invention;

FIG. 3 is a schematic block diagram of a synchronization apparatus of a coordinator according to an exemplary embodiment of the present invention;

FIG. 4 is a schematic block diagram of a synchronization apparatus of a device according to an exemplary embodiment of the present invention;

FIG. 5 is a flowchart of a synchronization method according to an exemplary embodiment of the present invention;

FIG. 6 is a schematic block diagram of a location awareness apparatus according to an exemplary embodiment of the present invention;

FIG. 7A illustrates a data frame according to an exemplary embodiment of the present invention;

FIG. 7B illustrates a data frame according to another exemplary embodiment of the present invention;

FIG. 8 illustrates an exemplary embodiment of the location awareness apparatus of FIG. 6;

FIG. 9 illustrates another exemplary embodiment of the location awareness apparatus of FIG. 6;

FIG. 10 is a timing diagram of clock signals output from a delay circuit of FIG. 9 according to an exemplary embodiment of the present invention; and

FIGS. 11A and 11B are graphs showing the results of simulation of a location awareness method according to an exemplary embodiment of the present invention.

### DETAILED DESCRIPTION OF EXEMPLARY EMBODIMENTS OF THE INVENTION

Hereinafter, exemplary embodiments of the present invention will be described in detail with reference the accompanying drawings.

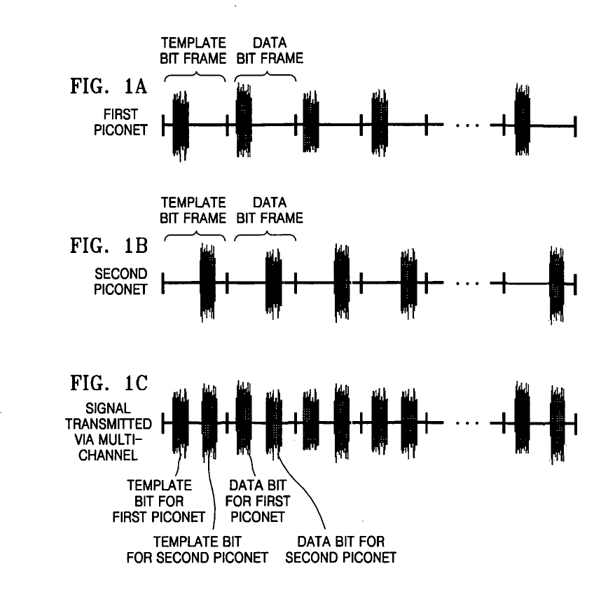

FIGS. 1A to 1C illustrate the structures of data frames used in a chaotic communication system according to an exemplary embodiment of the present invention. When there are a first piconet and a second piconet, a template unit of each data frame is divided into two pieces, a first piece is allocated to the first piconet, and a second piece is allocated to the second piconet.

Referring to FIG. 1A, a template bit for the first piconet is contained in the first piece of a template bit frame of a data frame of the first piconet, and at least data bit is contained in a first piece of each data bit frame of a data unit of the data frame.

Referring to FIG. 1B, a template bit for the second piconet is arranged in the second piece of a template bit frame of a data frame of the second piconet, and at least data bit is arranged in a second piece of each data bit frame of a data unit of the data frame.

Referring to FIG. 1C, the data frame of FIG. 1A overlaps with the data frame of FIG. 1B.

Therefore, even when a plurality of data frames are received via a multi-channel, if receiving apparatuses over the first piconet recognize that a desired template bit is arranged in a first piece of a template bit frame, they can detect the desired template bit from the first piece of the template bit frame and extract a data source signal from the detected template bit.

Likewise, if receiving apparatuses over the second piconet recognize that a desired template bit is arranged in a second piece of a template bit frame, they can detect the desired template bit from the second piece of the template bit frame and extract a data source signal from the detected template bit.

FIGS. 1A and 1B illustrate that a data bit is arranged in a piece of each data bit frame of a data frame, which corresponds to a piece of a template bit frame of the data frame. However, a chaotic communication system according to the present invention is not limited to the above illustration. In other words, although the template bit must be contained in a piece of the template bit frame, which is allocated to a piconet, the position of the data bit is not limited since it is possible to detect data using the template bit. For instance, all data bits may be contained in a data bit frame of the data frame.

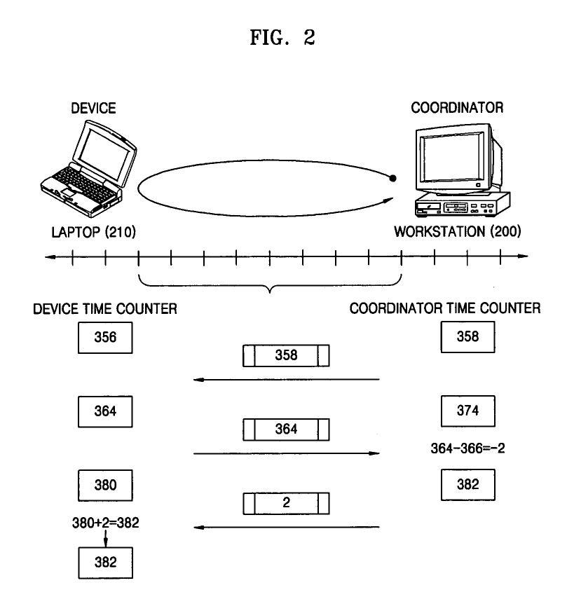

FIG. 2 is a reference diagram illustrating a synchronization method according to an exemplary embodiment of the present invention. Referring to FIG. 2, a workstation 200 which is an example of a coordinator is spaced by eight degrees of a scale from a laptop 210 which is an example of a device. It is assumed that a time required to move a packet by a degree of the scale is equal to a time required to increase a value of a coordinator time counter or a device time counter by one.

Initially, the coordinator time counter is set to a value of 358 and the device time counter is set to a value of 356, that is, the difference between the value of the coordinator time counter and the device time counter is 2. To cancel the difference, the workstation 200 transmits a predetermined packet to the laptop 210 for clock synchronization. The predetermined packet may or may not contain the value of the coordinator time counter determined when the packet is transmitted, but the determined value of the coordinator time counter must be memorized.

Referring to FIG. 2, when the value of the coordinator time counter is 358 and the value of the device time counter is 356, the workstation 200 transmits a predetermined packet to the laptop 210.

Since the laptop 210 is spaced from the workstation 200 by eight degrees, the laptop 210 receives the predetermined packet when the value of the device time counter increases by eight, i.e., when the value of 356 is increased to 364, and sends the workstation 200 a packet containing the value of 364 of the device time counter, the value of 364 being determined when the laptop 210 receives the predetermined packet.

Then, the workstation 200 receives the packet from the laptop 210 when the value of the coordinator time counter increases to 374. That is, the workstation 200 transmits the predetermined packet to the laptop 210 when the value of the coordinator time counter is 358, and receives the packet from the laptop 210 when the value of the coordinator time counter is 358+16=374, since the distance between the workstation 200 and the laptop 210 corresponds to eight degrees and a total distance from the workstation 200 to the laptop 210 and from the laptop 210 to the workstation 200 is 16 degrees.

The workstation 200 transmits the predetermined packet to the laptop 210 when the value of the coordinator time counter is 358 and receives the packet, which is a reply packet to the predetermined packet, from the laptop 210 when the value of the coordinator time counter is 374. Thus, the total distance from the workstation 200 to the laptop 210 and from the laptop 210 to the workstation 200 is 374−358=16 degrees, and half of 16 degrees, i.e., 8 degrees, is the distance between the laptop 210 and the workstation 200.

Since the distance between the workstation 200 and the laptop 210 is 8 degrees, the value of the device time counter, which is contained in the packet transmitted from the laptop 210 to the workstation 200, must be 358+8=366. However, the value of the device time counter, which is contained in the packet transmitted to the workstation 200, is 364. That is, the difference in values between the device time counter and the coordinator time counter is 364−366=−2.

To compensate for the difference of −2, the workstation 200 transmits a packet containing a value of +2 to the laptop 210 when the value of the coordinator time counter is 374.

Since a time required for the packet to be transmitted from the workstation 200 to the laptop 210 corresponds to 8 degrees, the laptop 210 receives the packet containing the value of +2 and increases the device time counter by +2 when the value of the device time counter becomes 364+16=380, thus the value of the device time counter is reset to 382.

The laptop 210 receives the packet from the workstation 200, which was sent when the value of the coordinator time counter is 374, after a time corresponding to 8 degrees has passed, and thus, the coordinator time counter is also reset to 374+8=382. Accordingly, the value of the device time counter can be synchronized with the value of the coordinator time counter at a predetermined instance of time.

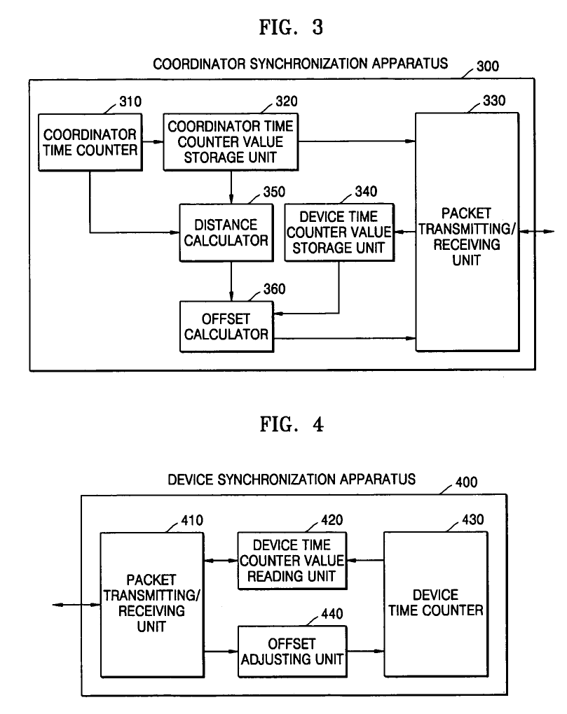

FIG. 3 is a schematic block diagram of a coordinator synchronization apparatus 300 according to an exemplary embodiment of the present invention. The coordinator synchronization apparatus 300 includes a coordinator time counter 310, a coordinator time counter value storage unit 320, a packet transmitting/receiving unit 330, a device time counter value storage unit 340, a distance calculator 350, and an offset calculator 360.

The coordinator time counter 310 counts coordinator time.

The coordinator time counter value storage unit 320 stores a coordinator time counter value determined when the coordinator transmits the predetermined packet to the device.

The packet transmitting/receiving unit 330 transmits the predetermined packet to the device or receives the predetermined packet from the device. According to the present invention, the packet transmitting/receiving unit 330 transmits a packet containing the coordinator time counter value or a predetermined packet to the device, receives a reply packet containing a device time counter value from the device, and sends the device a packet containing an offset to be used in synchronizing the device with the coordinator.

The device time counter value storage unit 340 stores the device time counter value received from the device.

The distance calculator 350 calculates the distance between the device and the coordinator by comparing the coordinator time counter value stored in the coordinator time counter value storage unit 320 with a coordinator time counter value determined when the packet transmitting/receiving unit 330 receives the device time counter value, according to the following equation:
Distance between device and coordinator=(coordinator time counter value when predetermined packet is transmitted to device−coordinator time counter value when device time counter value is received)×½

The offset calculator 360 calculates an offset using the distance computed by the distance calculator 350 and the device time counter value stored in the device time counter value storage unit 340 according to the following equation, and provides the calculated offset to the packet transmitting/receiving unit 330 so that it can be transmitted to the device.
Offset=device time counter value−(coordinator time counter value when predetermined packet is transmitted to device+distance)

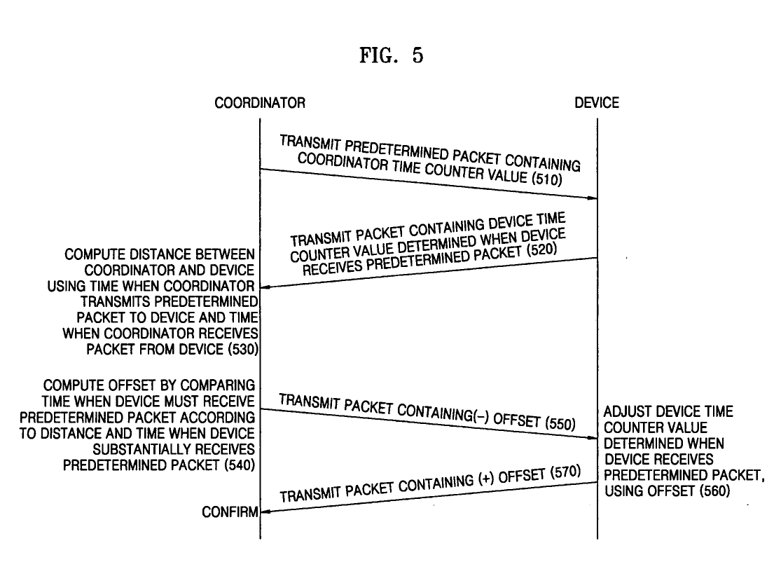

FIG. 4 is a schematic block diagram of a device synchronization apparatus 400 according to an exemplary embodiment of the present invention. The device synchronization apparatus 400 includes a packet transmitting/receiving unit 410, a device time counter value reading unit 420, a device time counter 430, and an offset adjusting unit 440.

The packet transmitting/receiving unit 410 receives a predetermined packet or a packet containing an offset from a coordinator (not shown) or transmits a packet containing a value of the device time counter 430 to the coordinator.

When the packet transmitting/receiving unit 410 receives the predetermined packet from the coordinator, the device time counter value reading unit 420 reads a device time counter value from the device time counter 430, which is determined when the predetermined packet is received, and provides the read device time counter value to the packet transmitting/receiving unit 410 so that it can be transmitted to the coordinator.

The device time counter 430 counts device time.

When the packet transmitting/receiving unit 410 receives a packet containing an offset from the coordinator, the offset adjusting unit 440 sends the offset to the device time counter 430 so that the offset is added to a value of the device time counter 430 is increased by the offset.

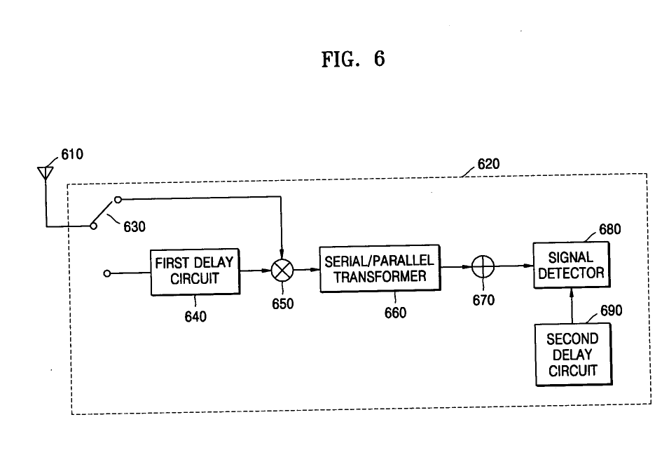

FIG. 5 is a flowchart of a synchronization method according to an exemplary embodiment of the present invention. Referring to FIG. 5, a coordinator transmits a packet containing a coordinator time counter value to a device (510). As described above, the packet need not include the coordinator time counter value, but the coordinator time counter value determined when the coordinator transmits the packet to the device must be memorized.

In receipt of the packet, the device sends the coordinator a reply packet which contains a device time counter value determined when the device receives the packet (520).

Then, the coordinator computes the distance between the coordinator and the device using time when the coordinator transmits the packet to the device and time when the coordinator receives the reply packet from the device (530). That is, the distance between the device and the coordinator may be computed by: (coordinator time counter value when coordinator transmits packet to device−coordinator time counter value when coordinator receives reply packet from device)×½.

Next, the coordinator computes an offset by comparing time when the coordinator should have received the reply packet and time when the coordinator substantially received the replay packet, using the distance computed in 530 (540). In other words, the offset may be computed by: device time counter value−(coordinator time counter value when the coordinator transmits the packet to the device+distance).

Next, the coordinator transmits a packet containing an (−) offset to the device (550).

Next, the device adjusts the device time counter value by adding the (−) offset to a device time counter value determined when the device receives the (−) offset from the coordinator (560).

The device may transmit a packet containing an (+) offset to the coordinator so as to inform the coordinator that the device safely received the (−) offset (570).

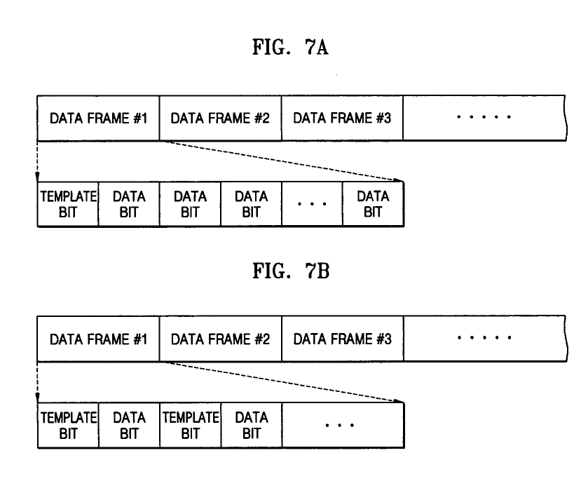

FIG. 6 is a schematic block diagram of a location awareness apparatus according to an exemplary embodiment of the present invention. The location awareness apparatus includes an antenna 610 and a signal processor 620.

The antenna 610 acts as a data frame receiver which receives a data frame from a communication network. That is, the antenna 610 receives a data frame from the communication network via at least one channel and transmits the data frame to a switch 630.

The signal processor 620 processes the received data frame to compute the distance between a data frame transmitting apparatus (not shown) and the location awareness apparatus.

The signal processor 620 includes the switch 630, a first delay circuit 640, a multiplier 650, a serial/parallel transformer 660, an adder 670, a signal detector 680, and a second delay circuit 690.

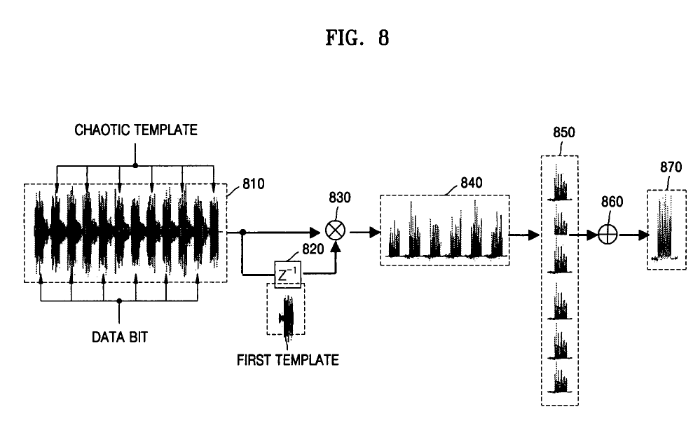

First, examples of a data frame according to an exemplary embodiment of the present invention will now be described with reference to FIGS. 7A and 7B. Referring to FIG. 7A, each data frame includes a template bit and a plurality of data bits. Referring to FIG. 7B, each data frame includes a plurality of template bit frames and a plurality of data bit frames.

According to the present invention, each data signal is divided into units of a predetermined data frame. Referring to FIGS. 7A and 7B, a data signal is comprised of a data frame #1, a data frame #2, a data frame #3, . . . .

Each data frame includes a template bit and a data bit. The template bit contains a template chaotic signal, and the data bit contains data modulated using the template chaotic signal. The template chaotic signal is a unique signal used in a predetermined data transmitting apparatus or a piconet to which the predetermined transmitting apparatus is connected. The data bit is a signal obtained by modulating a data source signal using the template chaotic signal.

The data frame #1 will now be described in greater detail with reference to FIG. 7A. Referring to FIG. 7A, the data frame #1 is comprised of a template bit and a plurality of data bits.

The template bit is a one-bit unit that contains a template chaotic signal. If the template bit is available to a plurality of users (or piconets), the template bit is divided into pieces and each piece is arranged at a predetermined position allocated to each user (or piconet). For instance, when there are four piconets, the template bit which is a one-bit unit is divided into four pieces, a first piece is allocated to contain a template for a first piconet, a second piece is allocated to contain a template for a second piconet, a third piece is allocated to contain a template for a third piconet, and a fourth piece is allocated to contain a template for a fourth piconet.

As described above, respective templates for different users or piconets are arranged in a template bit according to a predetermined order so that the templates do not overlap with one another. Thus, even if data frames are transmitted to a receiving side via a multi-channel, the receiving side can detect a desired template from a predetermined position of each data frame.

In contrast, respective data source signals for different users may be contained in the same position of a data bit. This is because even when data signals are contained in the same position of a data bit of a data frame and such data frames are transmitted to a receiving side via a multi-channel, the receiving side can exactly detect a desired data source signal by matching the detected data source signal with its template.

Referring to FIG. 7B, a data frame #1 contains plural pairs of a template bit and a data bit. That is, each data bit can have a corresponding template bit to increase the reliability of transmission of template bits.

Referring to FIG. 6, under the control of a controller (not shown), the switch 630 allows a template bit of a data frame input via the antenna 610 to be input to the first delay circuit 640, and a data bit of the data frame to be input to the multiplier 650.

The first delay circuit 640 stores template bits, and transmits each template bit to the multiplier 650 at a predetermined interval, that is, when a data bit is input to the multiplier 650. As described above, the positions of template bits, each being allocated to each apparatus, are known to the apparatuses, the first delay circuit 640 can detect a desired template bit from the data frame.

The multiplier 650 performs a multiplication operation on the data bit and the template bit, which is output from the first delay circuit 640, and provides the result of multiplication to the serial/parallel transformer 660. For instance, if a data frame includes 16 data bits, the multiplier 650 can perform the multiplication operation from one to sixteen times. In other words, the multiplier 650 may perform the multiplication operation on a first template bit and a first data bit only once and provide the result of multiplication to the serial-parallel transformer 660, or may perform the multiplication operation more than once and provide the results of operations to the serial/parallel transformer 660 so as to ease signal detection.

The serial/parallel transformer 660 receives serial data from the multiplier 650, transforms the serial data into parallel data, and provides the parallel data to the adder 670.

The adder 670 combines all data output from the serial/parallel transformer 660, and transmits the result of combination to the signal detector 680.

The signal detector 680 detects the envelope of a valid signal, rather than noise, from overlapped 16-bit signals, for example, and then detects a time when the envelope is generated, using the envelope. Next, the signal detector 680 detects the difference between the time when the envelope is generated, i.e., a template chaotic signal is substantially allocated due to a delay in signal transmission, with time when the template chaotic signal is to be originally allocated.

The time when the envelope is generated may be detected by computing moving averages of the signal and transforming an index representing a maximum value of the moving averages into time. The second delay circuit 690 is used to precisely detect the time when the envelope is generated. Compared to a conventional clock counter, the second delay circuit 690 includes a plurality of clock counters that provide precise clock signals and thus can enable precise location measurement even at a ultra-wide band.

According to the present invention, based on a fact that the position of a template chaotic signal for each user (or piconet) has been determined, a receiving side can detect the difference between the position at which the template chaotic signal is substantially contained and the position at which the template chaotic signal must be contained, and compute the distance between an apparatus transmitting a data frame and an apparatus receiving the data frame, using the difference.

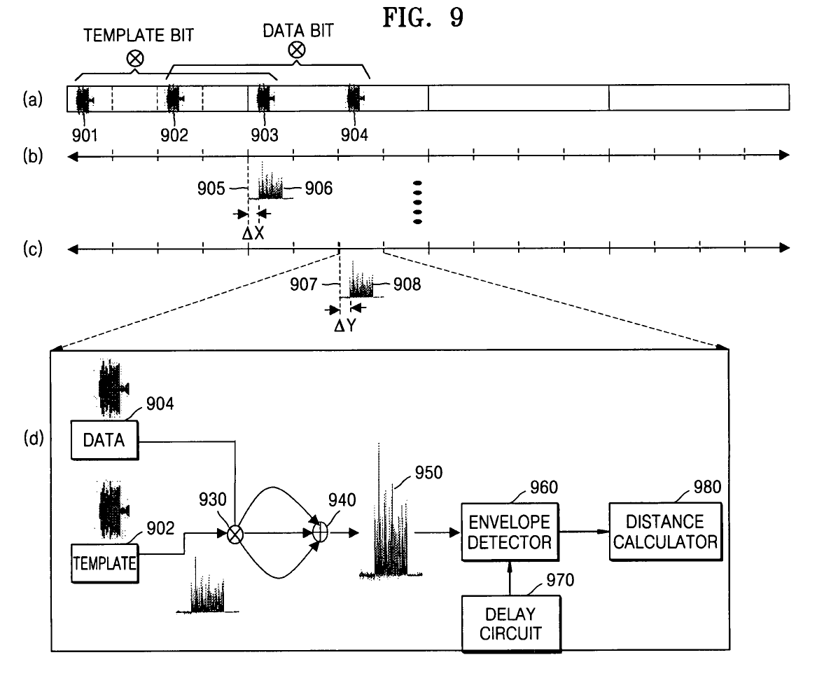

FIG. 8 illustrates an exemplary embodiment of the location awareness apparatus of FIG. 6. Referring to FIG. 8, the location awareness apparatus receives a data frame 810 which includes six chaotic templates and six data bits.

A delay circuit 820 receives and stores a first chaotic template from the data frame, and provides the first chaotic template to a multiplier 830 at a predetermined interval, i.e., every time when a data bit is input to the multiplier 830.

Upon receiving a data bit, the multiplier 830 generates six signals 840 in series by performing a multiplication operation on the data bit and a template signal output from the delay circuit 820.

The six serial signals 840 are transformed into six parallel signals 850, and the six parallel signals 850 are combined (or overlapped with one another) by an adder 860, thus obtaining a signal 870.

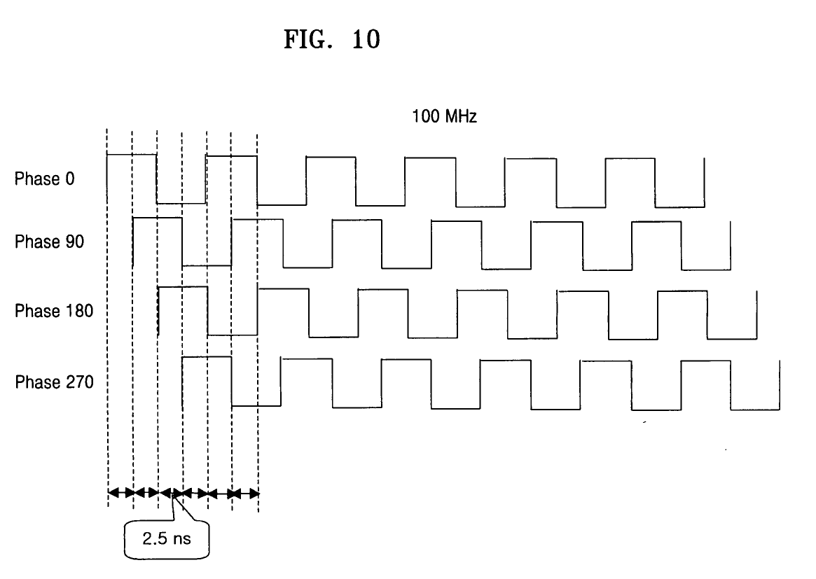

FIG. 9 illustrates another exemplary embodiment of the location awareness apparatus of FIG. 6. Specifically, (a) of FIG. 9 illustrates overlapped data frames input to the location awareness apparatus via two channels.

In FIG. 9, (b) denotes a signal 906 obtained by performing a multiplication operation on a template signal 901 and a data signal 903 which are allocated to a first user. ΔX denotes the difference between the position at which the signal 906 is substantially detected due to a delay in signal transmission and the position 905 at which the signal 906 must be detected.

In FIG. 9, (c) denotes a signal 908 obtained by performing the multiplication operation on a template signal 902 and a data signal 904 which are allocated to a second user. ΔY denotes the difference between the position at which the signal 908 is substantially detected due to a delay in signal transmission and the position 907 at which the signal 908 must be detected.

In FIG. 9, (d) is a schematic block diagram of a location awareness apparatus which detects the difference ΔY to determine the location of an apparatus that transmits the template signal 902 and the data signal 904. Referring to (d) of FIG. 9, the data signal 904 and the template signal 902 are transmitted to a multiplier 930, and the multiplier 930 generates the signal 908 by performing the multiplication operation on the template signal 902 and the data signal 904 and provides the signal 908 to an adder 940 three times. The reason why the signal 908 is provided to the adder 940 three times is to overlap the signals 908 with one another for ease detection of the signal 908.

The adder 940 combines all the signals 908 output from the multiplier 930 to obtain overlapped signals 950, and transmits the overlapped signals 950 to an envelope detector 960.

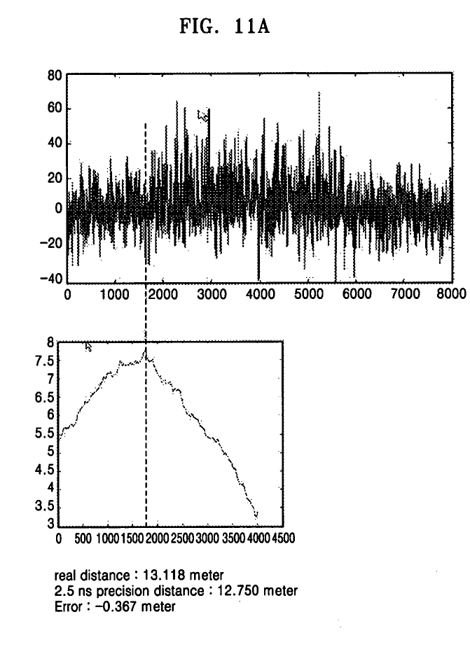

The envelope detector 960 detects the envelope of a valid signal, not noise, from the overlapped signals 950, and provides information regarding the envelope to a distance calculator 980. For precise detection of the envelope, the envelope detector 960 uses clock signals, such as those illustrated in FIG. 10, which are output from a delay circuit 970 FIG. 10.

FIG. 10 is a timing diagram of clock signals generated by the delay circuit 970 of FIG. 9. Referring to FIG. 10, the delay circuit 970 includes four clock counters of 100 MHz (not shown), and the four clock counters respectively generate the clock signals to be 90 degree out of phase with one another (i.e., Phase 0, Phase 90, Phase 180 and Phase 270 in FIG. 10). Next, the delay circuit 970 transmits a ¼ part of each clock signal, which corresponds to a rising edge of the clock signal, as a clock signal to the envelope detector 960, thereby enabling more precise clock control than when the entire clock signal is used. In other words, referring to FIG. 10, since the clock signals are generated using the four clock counters of 100 MHz, it is possible to detect an error of 2.5 ns in position between clock signals, i.e., detect the location of a device within the error range of about 1 m.

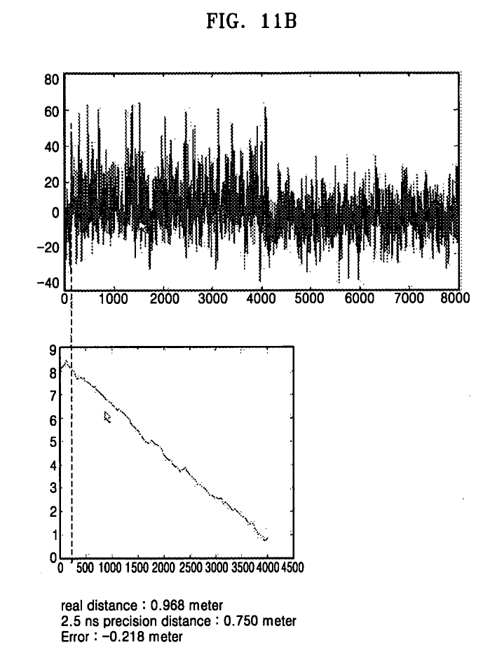

FIGS. 11A and 11B are graphs illustrating the results of simulation of a location awareness method according to an exemplary embodiment of the present invention. Here, each x-axis denotes the number of samples obtained for about 200 ns, and each y-axis denotes the amplitude of sample.

Specifically, the upper graph of FIG. 11A illustrates a signal, such as the signal 870 of FIG. 8 (or the signal 950 of FIG. 9), which was obtained by overlapping signals using an adder. The lower graph of FIG. 11A illustrates moving averages of the signal, wherein an index of the moving average between 1600 and 1700 samples is a maximum value, at which a valid signal, not noise, began to be generated. In the simulation result of FIG. 11A, a substantial distance between a transmitting side and a receiving side was 13.118 m, a distance measured with precision of about 2.5 ns according to the present invention was 12.750 m, and an error between the distances was −0.367 m.

The upper graph of FIG. 11B illustrates a signal, such as the signal 870 or the signal 950, which was obtained by overlapping signals with one another using an adder. The lower graph of FIG. 11B illustrates moving averages of the signal, wherein an index of the moving average of about 300 samples is a maximum value, at which a valid signal, not noise, began to be generated. In the simulation result of FIG. 11B, a substantial distance between a receiving side and a transmitting side was 0.968 m, a distance measured with precision of about 2.5 ns according to the present invention was 0.750 m, and an error between the distances was −0.218 m.

As described above, according to the present invention, it is possible to precisely synchronize with devices and measure the distance between the devices in a chaotic communication system.

While this invention has been particularly shown and described with reference to exemplary embodiments thereof, it will be understood by those skilled in the art that various changes in form and details may be made therein without departing from the spirit and scope of the invention as defined by the appended claims.

What is claimed is:

1. A synchronization method comprising:
calculating a distance between a first device and a second device based on a time when a first packet is transmitted from the first device to the second device and a time when a second packet containing a device time counter value transmitted by the second device in response to the first packet is received by the first device;
determining an offset by comparing the device time counter value in the second packet with a time when the second packet containing the device time counter value should be received by the first device according to the distance; and
providing the offset to the second device so that the second device adjusts the device time counter value based on the offset.

2. The synchronization method of claim 1, wherein the determining the offset comprises computing the offset by subtracting the device time counter value contained in the second packet from a sum of a device time counter value determined when the packet is transmitted and the distance.

3. The synchronization method of claim 1, wherein the first and second devices are devices of a chaotic communication system.

4. The synchronization method of claim 3, wherein the first device is a coordinator.

5. A synchronization method comprising:
receiving, at a first device, a packet from a second device;
transmitting a time counter value from the first device to the second device, wherein the time counter value is determined when the first device receives the packet;
receiving, at the first device, an offset from the second device, wherein the offset is determined by comparing the device time counter value with a time when the second device should receive the device time counter value according to a distance between the first device and the second device, and the distance is determined based on a time when the second device transmits the packet to the device and a time when the second device receives the device time counter value from the first device; and
adjusting the device time counter value of the first device based on the offset.

6. The synchronization method of claim 5, wherein the first and second devices are devices of a chaotic communication system.

7. The synchronization method of claim 6, wherein the second device is a coordinator.

8. A location awareness method in a chaotic communication system, comprising:
receiving a data frame which includes a template chaotic signal to be determined to be arranged at a predetermined position of the data frame allocated to a user, and a data source signal obtained by modulating the template chaotic signal;
detecting a position of the data frame in which the template chaotic signal is substantially arranged; and
determining a distance between a device transmitting the data frame and a device receiving the data frame based on a difference between the position of the data frame in which the template chaotic signal is substantially arranged and the predetermined position.

9. The location awareness method of claim 8, wherein the detecting the position of the data frame in which the template chaotic signal is substantially arranged comprises:
detecting the template chaotic signal from the data frame;
performing a multiplication operation on the detected template chaotic signal and at least one data source signal, and adding results of the multiplication operation; and
detecting from a result of the adding the position of the data frame in which the template chaotic signal is substantially arranged.

10. A synchronization apparatus in a chaotic communication system, the synchronization apparatus comprising:
a distance calculator which determines the distance between the synchronization apparatus and a device, using a time when a first packet is transmitted to the device and a time when a second packet containing a device time counter value in response to the first packet is received from the device;
an offset calculator which determines an offset by comparing the time counter value with a time when the device should receive the packet containing the device time counter value according to the distance; and
an offset transmitting unit which provides the offset to the device so that the device adjusts the time counter value based on the offset.

11. The synchronization apparatus of claim 10, wherein the offset calculator determines the offset by subtracting the device time counter value contained in the second packet from a sum of a time counter value determined when the first packet is transmitted to the device and the distance.

12. A synchronization apparatus of a device in a chaotic communication system, the synchronization apparatus comprising:
a packet transmitting unit which transmits a second packet containing a device time counter value determined when the device receives a first packet from another device; and
an offset adjusting unit which receives an offset from the other device and adjusts the device time counter value based on the offset,
wherein a distance is determined based on a time when the other device transmits the first packet to the device and a time when the other device receives the second packet containing the device time counter value from the device, and
the offset is determined by comparing the device time counter value and a time when the other device should receive the second packet containing the device time counter value according to the distance.

13. The synchronization apparatus of claim 12, wherein the other device is a coordinator.

14. A location awareness apparatus in a chaotic communication system, location awareness apparatus comprising:
a data frame receiving unit which receives a data frame which contains a template chaotic signal to be defined to be arranged at a predetermined position of the data frame allocated to a user, and a data source signal obtained by modulating the template chaotic signal; and
a signal processor which detects a position of the data frame in which the template chaotic signal is substantially arranged and determines a distance between a device receiving the data frame and a device transmitting the data frame by determining a difference between a position of the data frame in which the template chaotic signal is substantially arranged and the predetermined position.

15. The location awareness apparatus of claim 14, wherein the signal processor comprises:
a delay circuit which detects the template chaotic signal from the data frame;
a multiplier which performs a multiplication operation on the detected template chaotic signal and at least one data source signal;
an adder which adds results of multiplication operations output from the multiplier; and
a signal detector which detects, from a result of the adding, the position of the data frame in which the template chaotic signal is substantially arranged.
